(19) United States
(12) Patent Application Publication
(10) Pub. No.: US 2013/0052607 A1
(43) Pub. Date: Feb. 28, 2013

(54) DENTAL LIGHT DEVICE WITH IDENTIFICATION MEANS

(71) Applicants: Mark Gersh, Pasadena, CA (US); Marc Orloff, Glendale, CA (US); Jeff MacDonald, Pomona, CA (US)

(72) Inventors: Mark Gersh, Pasadena, CA (US); Marc Orloff, Glendale, CA (US); Jeff MacDonald, Pomona, CA (US)

(73) Assignee: KONINKLIJKE PHILIPS ELECTRONICS N.V., EINDHOVEN (NL)

(21) Appl. No.: 13/696,888

(22) PCT Filed: May 12, 2011
(86) PCT No.: PCT/US11/36286

§ 371 Date: Nov. 8, 2012

Related U.S. Application Data
(60) Provisional application No. 61/334,131, filed on May 12, 2010.

(51) Int. Cl. A61N 5/06

(52) U.S. Cl. 433/27

(57) ABSTRACT

This invention relates to a curing light device capable of curing dental composites with a means to blow air. The blown air is directed from the curing light onto the tooth that is exposed to the curing light. The blown air has the effect of cooling the surface of the tooth to maintain patient comfort and efficient curing of dental composites

### FIELD OF THE INVENTION

The present invention relates to any device suitable for photocuring, or photobleaching in general. Particularly, it relates a photocuring, or photobleaching device suitable for curing dental composites or acting on a whitening gel, respectively, having a fan to blow air to cool the tooth surface.

### BACKGROUND OF THE INVENTION

In the field of dentistry, tooth restoration and repaired, dental cavities are often filled and/or sealed with compounds that are photosensitive, either to visible and/or ultraviolet light. These compounds, commonly known as light-curable compounds, are placed within dental cavity preparations or onto dental surfaces and are cured when exposed to light from a dental curing light device.

Heat generated on the tooth surface by any curing lights during operation can be problematic. The radiometric power of light needed for the fastest cure times is enough to cause oral tissue to heat up. The higher the intensity of light, the more heat generated. Additionally, composites can also exotherm as they cure. In deep cavities where there is the need for the longest curing times and the most power, extensive dentin has been removed and decay removal can approach the pulpal chamber. Therefore, there is concern over damage to pulp. The industry standard used for pulpal temperature increase is less than 5° C. This same kind of problem is also encountered in photobleaching lights. Therefore, any efficient way of removing heat from the tooth surface is desirable for both curing and photobleaching lights.

Various ways have been attempted to manage the heat generated on the teeth from high intensity curing lights. Such methods includes limiting the exposure time and/or pause between exposure to allow the oral tissue to cool.

Another method is the operator attempting to prevent the high intensity curing light from directly contacting unprotected gingival, oral mucuous membrane or skin. However, this is often difficult as the user's vision of the cured area is often obstructed. The user is also unable to look at the curing area while the curing light is activated as the light is too bright to look at with the naked eye.

Another way to manage heat is to place wet cotton rolls on the tooth crown opposite the side of exposure to lessen temperature rise.

The curing light can also be used at low power levels to reduce heat generation. While low power levels reduce heat, curing times may be increased and curing perforamce may be reduced due to the lower power levels.

All of the approaches listed above have drawbacks associated with them. Such drawbacks includes extending the time it takes to complete the procedure, increase patient discomfort, increase the risk of patient injury and decreasing the efficacy of the composite cure.

Therefore, there remains a need for a device that will effectively manage or remove heat from the tooth surface while maintaining high intensity power levels to ensure efficient curing or photobleaching of compounds.

### SUMMARY OF THE INVENTION

The present invention relates to a portable dental curing light suitable for curing light curable dental composite materials with a means for cooling the surface of the teeth. The curing light device may be “pen” style, “gun” style or convertible between “pen” and “gun” style. The curing light will include a housing having a handle portion towards its distal end, a front portion towards its proximal end, and a light module inside the front portion. The light module houses one or more light sources, which may include, for example, light emitting diodes (LEDs). The LEDs may emit wavelengths having a single or multiple peaks, one or both is in the range of most photosensitizers used in the curable composites such as dental composites. Also located in the device is a fan or other air circulation device which blows air out through the tip of the device, thereby cooling the tooth surface.

The housing of the curing light may be substantially cylindrical, with a slight taper from the distal end to the proximal end, for example. The proximal end of the housing may be straight or may be angled. In addition, the housing may have a vent leading to an air channel so that air is pulled into the housing by the fan. As air passes through the fan, it passes through another air channel that leads to the exhaust vent, which is proximate to the tip. Air exists the tip and contacts the tooth surface and has a cooling effect on the tooth surface.

The housing of the curing light may have a substantially hollow interior with at least one heat sink located therein, as discussed above. The heat sink may take on various shapes, and/or may include at least one phase change material, some of which may facilitate the arrangement of the light sources for a longer runtime device.

The curing light includes a light module housing having a distal end and a proximal end. The light module housing may have a substantially cylindrical shape defining a substantially hollow interior, a handle, a head and neck portion, with at least one elongated heat sink located therein. The head portion may be angled with respect to the rest of the housing. At least one mounting surface is located towards the proximal end of the elongated heat sink. An air channel with runs through the light module housing with an exit vent located proximate the head portion.

Light sources may include semiconductor light emitting devices, light-emitting chips such as an LED, a solid state LED, an LED array, edge emitting chips, a fiber optic bundle and so on.

A thermistor may also be present to provide temperature control of the light source wherein one end of the thermistor is attached to a control circuit board, and the other end may be inserted into the heat sink. When the temperature of the LEDs is higher than certain set point of the thermistor, a signal to shut off the curing light may be transmitted to the control circuit board to cool down the curing light for a period of time, which may not only prevent the curing light device from overheating, but may also protect patients from discomfort.

In one embodiment, the mounting surface may include an optical element, which may be concave or convex, and for directing and/or focusing light from a light source to a desired location, such as the mouth of the patient. In one aspect, the optical element may include lenses that are individually aligned with the corresponding LEDs to direct and/or focus light therefrom, to generate a high intensity round beam with less heat dissipation.

In one aspect, a heat sink made of a solid block of thermally conductive material, such as metal, may be used to efficiently remove or divert heat from a light source or sources. In another aspect, the heat sink may be configured to have fins, corrugations, or other geometric features adapted to provide a larger surface area for convective cooling of the heat sink. In a further aspect, the heat sink may include a substantially hollow interior which may be partially filled with at least one suitable phase change material including organic materials, inorganic materials and combination thereof, as noted before. These materials can undergo substantially reversible phase changes, and can typically go through a large, if not an infinite number of cycles without losing their effectiveness. A capping device may be used to cap off the heat sink after filling with the phase change material. The capping device may be compression fit. Any fitting may be sufficient to withstand any expansion and/or contraction force during cycling of the phase change material. In still another aspect, the heat sink may be constructed by hollowing out a thermally conductive material, such as metal, and at least partially filling the space with at least one phase change material prior to capping it to secure the phase change material inside, such that at least one phase change material is substantially contained or surrounded by a thermally conductive material such as a metal normally used in the construction if a conventional metal heat sink.

The light module housing may have a substantially cylindrical shape defining a substantially hollow interior, a handle, a head and neck portion, which may be angled with respect to the rest of the housing. At least one mounting surface is located towards the proximal end of the elongated heat sink. The light source may be located at the distal end of the heat sink. Within the housing is a fan. The fan is connected to an intake channel which draws air from the exterior to the fan, and an outlet channel which sends air from the fan to the exit vent. The exit vent is angled such that air leaving the exit vent is directed to the tooth surface.

The curing light of the present invention is compact, light weight, cordless and portable.

Any of the curing lights described above may provide light at single or multiple wavelengths commonly used for restorative compounds, as noted above. In one aspect, for multiple wavelengths, the light source may emit multiple wavelengths. In another aspect, the light source may emit one wavelength and the optical element may include a wavelength transformer having a chemical capable of absorbing the incident light and emitting light having a longer wavelength.

The curing light may be un-tethered and powered by, for example, a portable energy source, such as a battery, capacitor and/or combinations thereof. The battery can be removable or non-removable. A charger may be provided for charging the portable energy source during off cycle. In one aspect, the charger base may include an electric motor mechanically coupled to a fan or turbine. The fan or turbine may be adapted to draw or urge ambient air across a surface of the heat sink to provide cooling of the heat sink. In one embodiment, this cooling may occur when the curing light is at rest or being recharged. In another embodiment, the cooling means is present inside a charger base or cradle, for recharging the curing light. In other embodiments, the charger base or cradle may not have a fan or cooling means, but instead or additionally, many include a display panel for displaying a condition of the battery.

Another embodiment of this invention is a curing light device which can convert between a “pen” style curing light and a “gun” style curing light. In this embodiment, the handle itself can switch configurations. An alternative embodiment will have interchangeable handles for the various configurations.

Yet another embodiment of this invention is removable smart tips. The tips that are attached to the curing light devices have a variety of parameters, such as length, diameter, curvature and shape, which affects the light output. The removable smart tips transmit parameter information to the curing light device. The curing light device is able to use the information transmitted to it and adjust the light output so that each removable smart tip outputs the same level of light. This embodiment ensures that the curing light is emitting the proper intensity of light for each particular use. Also, but varying the intensity of light, battery life is improved.

The present invention together with the above and other advantages may best be understood from the following detailed description of the embodiments of the invention illustrated in the drawings below.

### BRIEF DESCRIPTION OF THE DRAWINGS

FIG. 1 illustrates a perspective view of a curing light of the present invention.

FIG. 2 illustrates an exploded view of the curing light of the present invention.

FIG. 3 illustrates a sectional view of the curing light of the present invention.

FIG. 4 illustrates cross-sectional views of a light module of the curing light in the present invention.

FIG. 5 illustrates a cross-sectional view of the fan module in the present invention.

FIG. 6 illustrates a cooling fan of the present invention.

FIG. 7 illustrates a perspective view of the lens cap engaging with the light emitting end with a pair of engaging units.

FIG. 8 illustrates a perspective view of the curing light with a pulse circuit.

FIG. 9 illustrates the pulse circuit electrically connecting with the thermistor and the LED circuit.

FIGS. 10 and 11 illustrate a charger base of the curing light in the present invention.

FIG. 12 illustrates an exploded view of the heat sink and portion of the light module attached to the heat sink.

FIG. 13 illustrates a perspective posterior view of the handle portion of the curing light in the present invention.

FIG. 14 illustrates a perspective anterior view of the handle portion of the curing light in the present invention.

FIG. 15 illustrates a perspective view of a “gun” style curing light of the present invention.

FIGS. 16a-c illustrates a perspective view of a curing light of the present invention which is capable of being converted from a “pen” style curing light to a “gun” style curing light.

FIGS. 17a-c illustrates a perspective view of a curing light of the present invention which is capable of being converted from a “pen” style curing light to a “gun” style curing light.

FIG. 18 illustrates a perspective view of a removable smart tip.

FIG. 18a illustrates a series of removable smart tips contained in a kit.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described.

All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

A curing light device useful for curing or activating light-activated materials is disclosed. The present invention has applications in a variety of fields, including but not limited to medicine and dentistry, where light-activated materials including a photoinitiator or photoinitiators are used. As an example, a photoinitiator absorbs light of a particular wavelength and initiates the polymerization of monomers into polymers.

In an exemplary embodiment, light-activated materials including a single photoinitiator or multiple photoinitiators may be applied to a surface, such as a tooth surface, and later cured by light of a wavelength or wavelengths that activates or activate the photoinitiator or photoinitiators. The light used is not only of a wavelength to which the photoinitiator is sensitive, but also of a power level adapted to cause curing over certain durations of time. Although the light used to activate the photoinitiator is of a wavelength to which a photoinitiator is sensitive, the light may come from a variety of sources, for example, a lamp, an arc lamp such as a halogen light source, semiconductor light emitting devices, light-emitting chips such as an LED, a solid state LED, an LED array, a fluorescent bulb, a fiber optic bundle and so on. Further for example, the present invention comprises light sources including semiconductor chips, LED dies, solid state LEDs, LED arrays, edgec emitting chips, or combinations thereof. The light source may include an emitting surface or at least one emitting edge as in the case of an edge emitting chip noted above, for a compact curing light device.

The typical sensitizers used in composite curing include Camphorquinone (CQ), which absorbs at about 465 nm and phenyl-propanedione (PPD), which absorbs at about 390 nm. Dental curing lights having multiple wavelengths suitable for curing curable composites usually comprise output wavelengths encompassing both of the absorbing wavelengths of these two typically used photo-initiators. The output wavelengths generally include a composite spectrum generated by LEDs or LED arrays emitting different wavelengths. The present invention comprises a curing light capable of curing all typical dental composites using, for example, light sources mentioned above, including semiconductor chips, LED dies, solid state LEDs, LED arrays, edge emitting chips, fiber optic bundles or combinations thereof, mounted on mounting platforms configured on at least one heat sink.

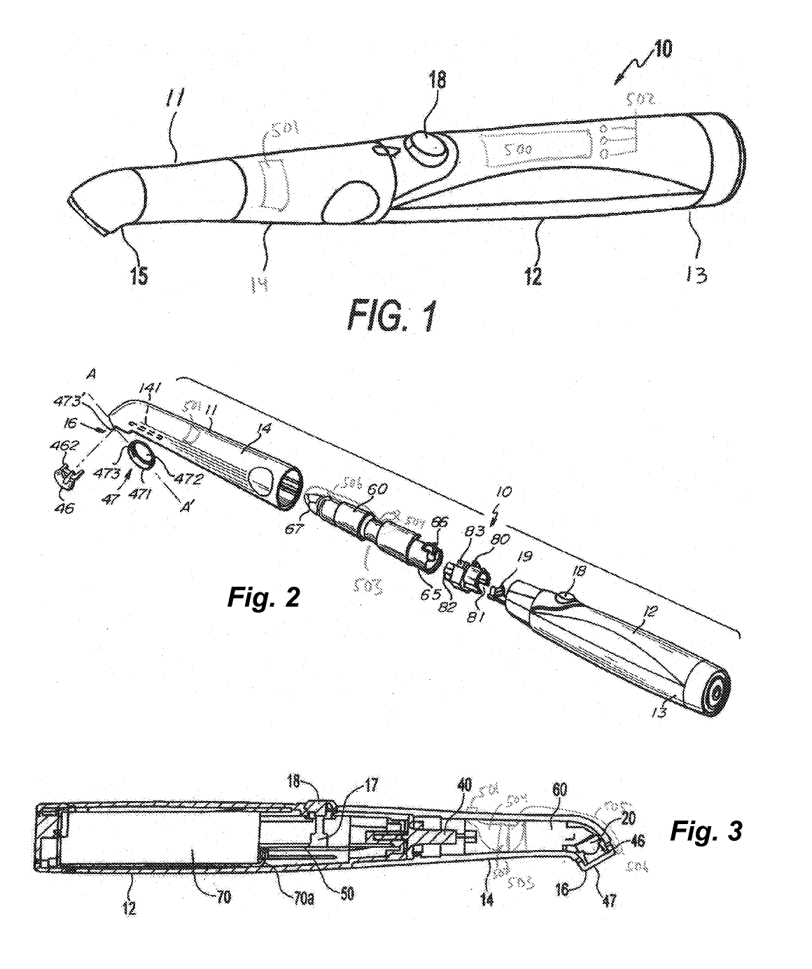

According to one embodiment, as illustrated in FIG. 1, 2 or 3, a handheld curing light 10 of the present invention includes a longitudinal housing having a distal end 11 and a proximal end 13 with a substantially hollow interior. In the present embodiment, the housing may include two portions, as depicted in the figures, a handle portion 12 and a front portion 14. LCD screen 500 displays status updates for the procedures, as well as permits the user to control different functions of the handheld curing light 10 if LCD screen 500 is a touchscreen. In the alternative, function buttons 502 permits the user to control different functions of the curing light. Intake vents 501 are connected to the intake channel 504 as seen in FIG. 3. It is noted, however, that a one-part housing may also be contemplated as part of the present invention. The front portion 14 may also be an extension of the housing, especially if an integral housing is present.

The portions 12 and 14 may be joined together by any attachment means, with the proximal end of handle portion 12 abutting the distal end of the front portion 14. Suitable attachment modes include, but are not limited to, friction fit, mating bayonet formations, tongue and groove type formations, interesting pin and pinhole formations, latches and other interconnecting structures.

As shown in FIG. 1, front portion of housing 14 of the curing light of the present invention also has a neck section 15, and this neck portion may be configured such that an emitting end 16 substantially coincides with the terminal end of the mounting deck, surface, platform or member of the light source 20, as shown in FIG. 3. The neck portion may also be angled with respect to the longitudinal portion of the housing. As shown in FIG. 3, the fan module 503 is located within the curing light device 10. Fan module can be located in either front portion 14 or handle 12. Air is drawn into the curing light device 10 through the intake vents 501 through the intake channel 504 into the fan module 503. Fan module 503 blows the air through output channel 505 where the air exits the curing light device through exit vent 506.

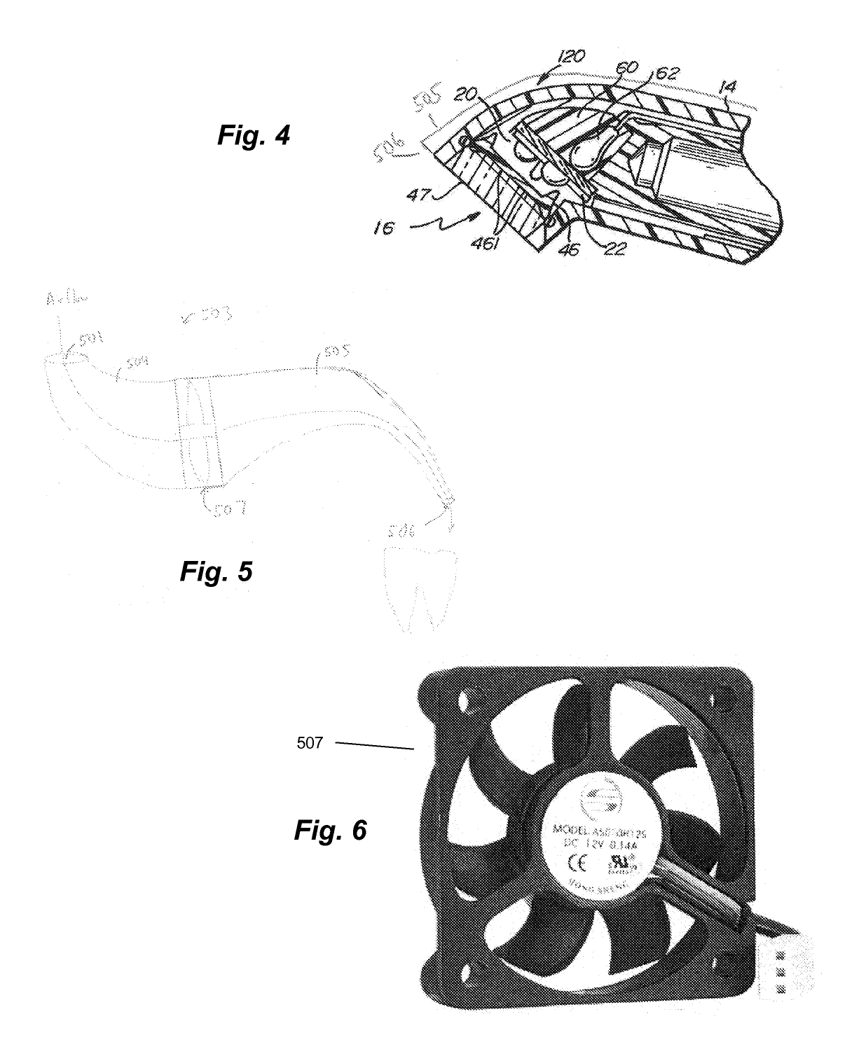

In one embodiment, as shown, for example, in FIG. 4, the front portion of the housing 14 may include a light module 120 in a desirable position in the interior of the front housing portion 14. The light module may include at least one light source 20, which, for example, may include LEDs or a fiber optic bundle. In another embodiment, the lens 461 may be inter-connected to form a continuous structure. At least one heat sink 60 may be located inside the light module 120 to conduct heat away from the light source 20. The proximal end 14 of the housing may further include a lens cap 47, which may be transparent, as exemplified, to provide an exit aperture for light from light source 20 and to serve as a protective cover to close the light emitting end 16 of the curing light. The lens cap 47 may also include other optical properties for focusing the light, etc., as discussed above, and in more detail below. Output channel 505 is proximate to the light module 120. Output channel 505 can be located on the exterior or interior of the housing portion 14 and is connected to the fan module 503.

The optical element 46 may be made of any substantially transparent material, including materials such as polycarbonate (Lexan®), polyacrylics, or any of the materials mentioned below that is substantially transparent.

FIG. 2 shows fan module 503. Air enters the intake channel 504 through intake vent 501. There is going to be at least one intake vent 501, although, multiple intake vents 501 can lead to the same intake channel 504 to increase the amount of air flowing to the fan. Air is pulled into the intake vent 501, through the intake channel 504 by the fan 507. After the air passes through the fan 507, it is pushed through output channel 505 and leaves the fan module 503 through exit vent 506 towards the tooth surface. There is at least one exit vent 506, although, multiple output vents 506 can be utilized.

FIG. 6 shows a fan 507. Fan 507 can be any standard cooling fan known to those skilled in the art that will fit within the fan module 503. At least one fan 507 will be mounted within the fan module 503, however, multiple fans 507 can be utilized depending on the size of fan module 503. Fan module 503 will be located in either the handle portion 12 or a front portion 14. Although, it is preferred that the fan module 503 be located as close to the distal end 11 of the curing light device 10 as possible, so the air will not need to travel as far before contacting the tooth surface. Fan 507 should be able to move enough air to cool the tooth surface such that the temperature of the tooth surface does not increase by more than 5° C.

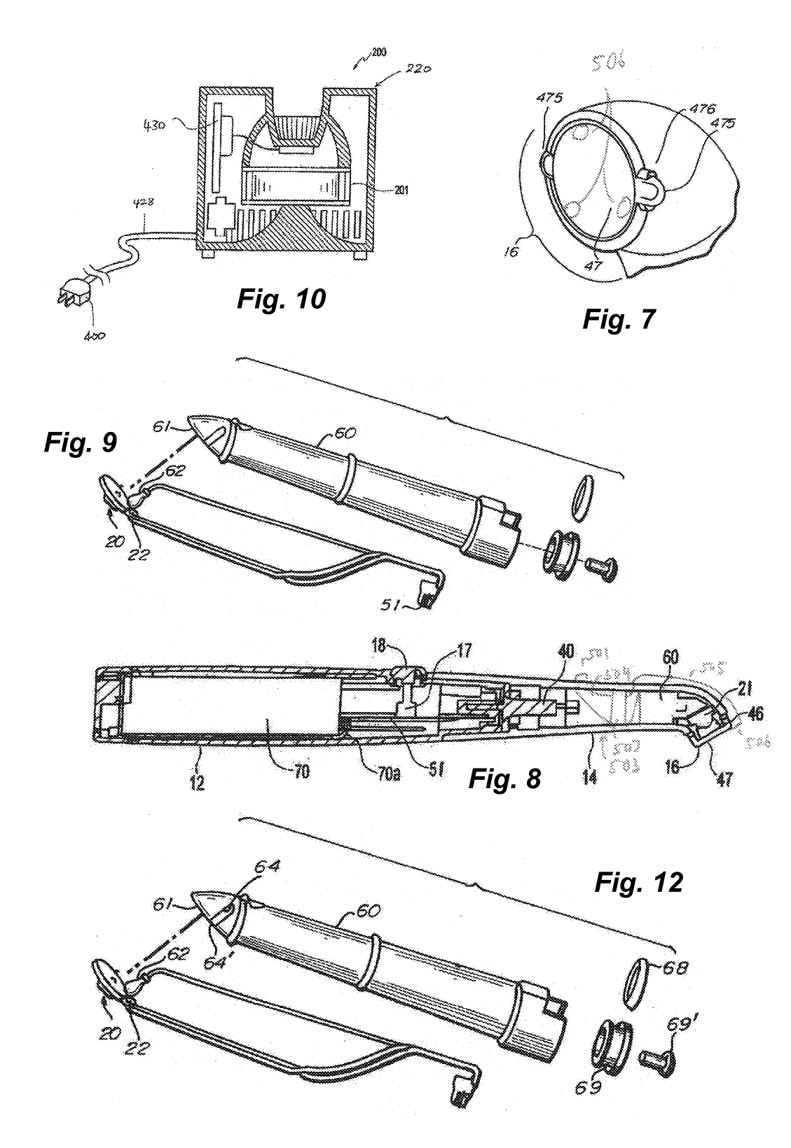

FIG. 7 is an embodiment with three exit vents 506. However, any number of exit vents 506 can be utilized in different arrangements. FIG. 7 also shows a removable lens cap 47. In this embodiment, lens cap 47 may include a pair of engaging units 475 symmetrically located at the periphery of the lens cap 47, to be connected to a resilient material, so that the user may slightly press the engaging units 475 inwardly to engage the receiving slot 476 located at the light emitting end 16. However, the actual attachment and detachment mechanism for removable lens cap 47 can be any number of ways known to those skilled in the art.

In one embodiment, the heat sink 60 may be elongated and positioned inside the front portion 14, in close-proximity to the light source 20, to conduct, or dissipate heat there from. In one aspect, as illustrated in FIG. 4, the light source 20 (on the substrate 22) is attached or glued, with thermal adhesive including thermosetting or structural adhesives, to the mounting surface 61 of the heat sink 60. A thermistor 62 may also be attached or glued to the outer surface of the heat sink 60 with the same material noted above, wherein one end of the thermistor 62 may be attached to the control circuit board 50 to control on/off of the curing light 10, and the other end may be inserted into the heat sink 60. When the temperature of the LEDs is higher than certain set points of the thermistor, a feedback signal may be generated and transmitted to the control circuit board 50 to shut off the curing light device 10 for a period of time to bring the temperature down to the set point. This feedback control scheme not only prevents the curing light device 10 from overheat, but also protects patients from discomfort.

In one embodiment, the optical element 46 and the front portion 14, or at least portions of the front portion 14 may be, for example, made out of the same material, similar material, or different material having little or no difference in the coefficients of thermal expansion. With the presence of different coefficients of thermal expansion, hoop stress may result, which may lead to premature failure of the unit. Such failure is minimized or eliminated by the present embodiment of the invention.

For example, a polymer useful in the present invention may be a polymer that may be molded or cast. Suitable polymers include polyethylene, polypropylene, polybutylene, polystyrene, polyester, acrylic polymers, polyvinylchloride, polyamide, or polyetherimide like ULTEM®; a polymeric alloy such as Xenoy®. resin, which is a composite of polycarbonate and polybutyleneterephthalate or Lexan®. plastic, which is a copolymer of polycarbonate and isophthalate terephthalate resorcinol resin (all available from GE Plastics), liquid crystal polymers, such as an aromatic polyester or an aromatic polyester amide containing, as a constituent, at least one compound selected from the group consisting of an aromatic hydroxycarboxylic acid (such as hydroxybenzoate (rigid monomer), hydroxynaphthoate (flexible monomer), an aromatic hydroxyamine and an aromatic diamine, (exemplified in U.S. Pat. Nos. 6,242,063, 6,274,242, 6,643,552 and 6,797,198, the contents of which are incorporated herein by reference), polyesterimide anhydrides with terminal anhydride group or lateral anhydrides (exemplified in U.S. Pat. No. 6,730,377, the content of which is incorporated herein by referfence) or combinations thereof.

In addition, any polymeric composite such as engineering prepregs or composites, may also be used. For example, a blend of polycarbonate and ABS (Acrylonitrile Butadiene Styrene) may be used for the housing. Generally, materials usable in housing include, for example, polymeric materials or composites having high temperature resistance.

A liquid crystal polymer or a cholesteric liquid crystal polymer, such as one that can reflect rather than transmit light energy, may be used in various embodiments of the invention. (For example, in U.S. Pat. Nos. 4,293,435, 5,332,522, 6,043,861, 6,046,791, 6,573,963, and 6,836,314, the contents of which are incorporated herein by reference).

The lens cap 47 is generally transparent, as noted above. Thus, any material that can produce a transparent lens cap may be used.

The lens cap 47 may in general be disposable, so that it may be replaced when it becomes too dirty or has too many scratches on the surface to assure the quality and intensity of the light exiting from the lens cap 47.

Referring to FIG. 2, the curing light 10 may further include an internal connector 80 which is adapted to connect the handle portion 12 and the heat sink 60. In one embodiment, the handle portion 12 engages with the distal end 81 of the connector 80, while the heat sink 60 engages with the proximal end 82. Furthermore, a control circuit board 50 adapted to control the substrate 22 is received in the hollow interior 81 of the internal connector 80.

It is worth mentioning that the connector 80 may also assist the alignment between the substrate 22 and the optical element 46 by limiting the movement of the heat sink 60. For example, the proximal end 82 of the connector 80 may include an opening 83, which are received in an engaging portion 65 of the heat sink 60. The engaging portion 65 may also include an opening 66 and when the connector 80 engages with the heat sink 60, the openings 66 and 83 are aligned with each other, as shown in FIG. 2. When the heat sink 60 slightly rotates to misalign the openings, the heat sink 60 disengages the connector 80. In other words, the rotational movement of the heat sink 60 is so limited when engaging the connector 80, such that the precision of the alignment of between the substrate 22 (located at the mounting surface 61 of the heat sink 60) and the optical element 46 is achieved.

In one aspect, a guiding slot 67 is located at the side wall of the front portion of the heat sink 60. The guiding slot 67 matches with a guiding protrusion 141 inside the front portion 14, such that the substrate 22 at the heat sink 60 can be located at the appropriate position when slid onto the guiding protrusion 141, and thus precisely align with the optic element 46.

In one aspect, a pulse circuit 51 may provide electrical current to the LEDs in a pulsed manner, meaning that the current is steadily provided for a period of time, and then rest for a period of time, etc, as shown in FIG. 8. When the current is consistently provided to the LEDs, the heat is likely to buildup to decrease the intensity of the lights. If the electrical current is input into the LEDs in a pulsed manner, the heat can be dissipated or released at the time without electrical current. This pulsed current input scheme is surmised to improve the efficiency of heat dissipation, and thus increase the runtime and intensity of the curing light device 10.

In another aspect, the pulse circuit 51 may include a microprocessor, for controlling the on/off cycle, or the input or output power level.

In still another aspect, the pulse circuit 51 may include an input for receiving signals from the thermistor 62 and an output for controlling the intensity of the LEDs to minimize heat generation, as shown in FIG. 5a. More specifically, when the temperature of the LEDs is higher than certain set point of the thermistor 62, a feedback signal may be generated and transmitted to the pulse circuit board 51 to provide a pulse current to the LEDs to minimize the heat generation, as discussed above, and to further increase the runtime of the curing light device 10. It is surmised that the intensity of the curing light device 10 may be increased as well with pulsing.

In a further aspect, the pulse circuit 51 may monitor the output power level of the LEDs in a similar manner as illustrated above, and make corresponding adjustment of the electrical current to minimize heat generation, and further improve the runtime of the curing light device 10.

The pulsing is not limited to on and off, but to different levels of input power or output power. For example, a rest cycle may give off one half of the power of the on cycle, etc. In another example, the pulsing may be a half sinusoidal signal, like a bell-shape curve, going from zero to a maximum and down to zero again, etc.

Generally speaking, the heat sink 60 may be constructed by hollowing out a thermally conductive material, such as metal, and at least partially filling the void with at least one phase change material prior to capping it to secure the phase change material inside, such that the at least one phase change material is substantially contained or surrounded by a thermally conductive material such as metal normally used in the construction of a conventional heat sink.

In another embodiment, the heat sink may include a block of thermally conductive material such as metal having a bore or void space which is at least partially filled with a phase change material.

In a further embodiment, the heat sink 60 may be configured to have fins, corrugations, or other geometric features adapted to provide a larger surface area for convective cooling of the heat sink, whether the heat sink is a solid metallic block, partially filled with a phase change material, so on.

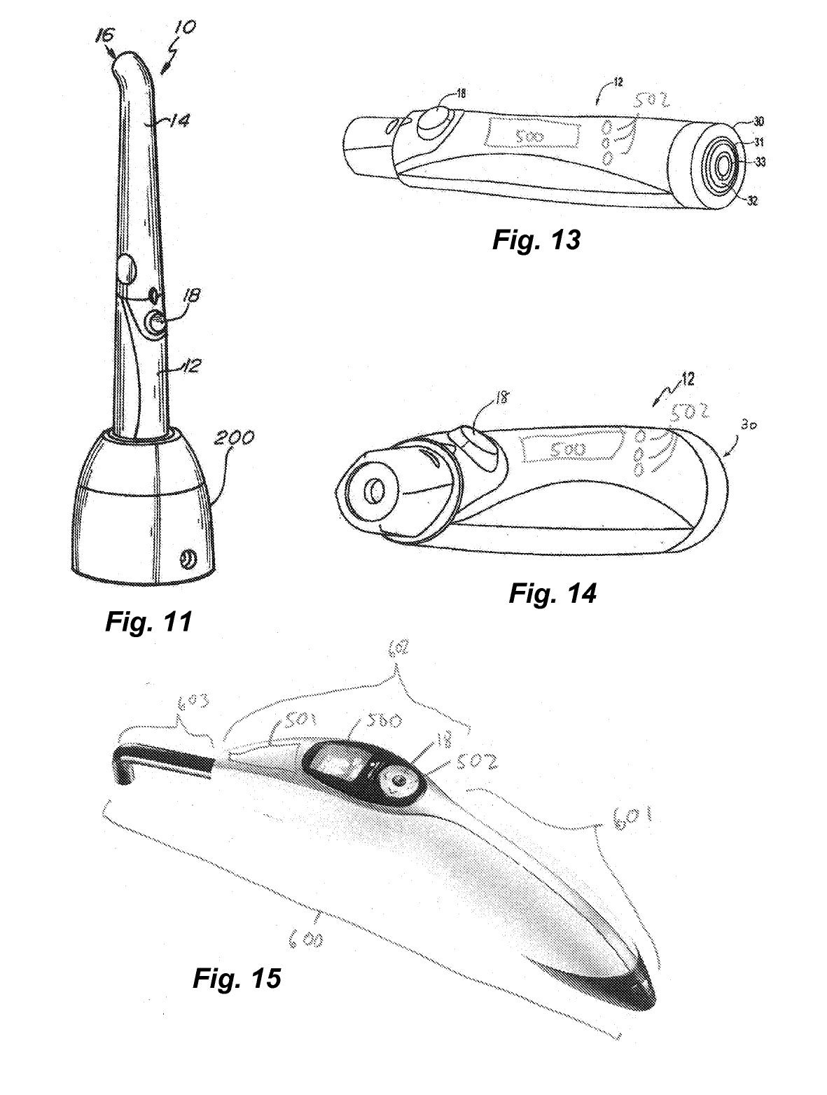

In still another embodiment, the curing light device 10 may include an electric motor mechanically coupled to a fan or turbine (as shown in FIGS. 10 and 11). The fan or turbine may be adapted to draw or urge ambient air across a surface of the heat sink 60 to provide cooling effect thereof.

The heat sink 60 may be made of any material that has good thermal conductivity, and/or dissipation properties, such as a metal or non-metal, for example, copper, aluminum, silver, magnesium, steel, silicon carbide, boron nitride, tungsten, molybdenum, cobalt, chrome, Si, SiO₂, SiC, AlSi, AlSiC, natural diamond, monocrystalline diamond, polycrystalline diamond, polycrystalline diamond compacts, diamond deposited through chemical vapor deposition and diamond deposited through physical vapor deposition, and composite materials or compounds.

Exemplary phase change materials are generally solid at ambient temperature, having melting points between about 30° C. and about 50° C., or between about 35° C. and about 45° C. Also, the exemplary materials may have a high specific heat, for example, at least about 1.7, more for example, at least about 1.9, when they are in the state at ambient temperature. In addition, the phase change materials may, for example, have a specific heat of at least about 1.5, more for example, at least about 1.6, when they are in the state at the elevated temperatures.

Some of the phase change materials mentioned above may be recyclable in that they may undergo phase changes for an almost infinite number of times. Others may be more endothermic agents and thus may have a limited life cycle unless handled under a controlled environment. These endothermic agents may lose their effectiveness as a phase change material even when handled under a controlled environment.

Thermal conductivity of the materials is a factor in determining the rate of heat transfer from the thermally conductive casing to the phase change material and vice versa. The thermal conductivity of the phase change material may be, for example, at least about 0.5 W/m° C. in the state at ambient temperature and at least about 0.45 W/m° C. in the state at elevated temperature.

Heat sinks having a phase change material may more efficiently remove or divert heat from a light source or sources with a given weight of heat sink material when compared to a heat sink made of a solid block of thermally conductive material such as metal. Such a heat sink may even efficiently remove or divert heat from a curing light device when a reduced weight of the material is used. Using a phase change material enclosed inside a hollow thermally conductive material such as a metal heat sink instead of a conventional solid metal heat sink can decrease the weight of the curing light and increase the time the heat sink takes to reach the “shut off” temperature, as it is called in the dental curing light industry. The period prior to reaching the shut off temperature is called the “run time”. Increasing the “runtime”, i.e., the time that the light can remain on, increases the time when a dentist can perform the curing or whitening procedure.

Suitable phase change material may include organic materials, inorganic materials and combinations thereof. These materials can undergo substantially reversible phase changes, and can typically go through a large, if not an infinite number of cycles without losing their effectiveness. Organic phase change materials include paraffin waxes, 2,2-dimethyl-n-docosane (C.sub.24H.sub.50), trimyristin, ((C.sub.13H.sub.27COO).sub.3C.sub.3H.sub.3), and 1,3-methyl pentacosane (C.sub.26H.sub.54). Inorganic materials such as hydrated salts including sodium hydrogen phosphate dodecahydrate (Na.sub.2HPO.sub.4.12H.sub.2O), sodium sulfate decahydrate (Na.sub.2SO.sub.4.10H.sub.2O), ferric chloride hexahydrate (FeCl.sub.3.6H.sub.2O), and TH29 (a hydrated salt having a melting temperature of 29.degree. C., available from TEAP Energy of Wangara, Australia) or metallic alloys, such as Ostalloy 117 or UM47 (available from Umicore Electro-Optic Materials) are also contemplated. Exemplary materials are solids at ambient temperature, having melting points between about 30.degree. C. and about 50.degree. C., more for example, between about 35.degree. C. and about 45.degree. C. Also, the exemplary materials have a high specific heat, for example, at least about 1.7, more for example, at least about 1.9, when they are in the state at ambient temperature. In addition, the phase change materials may, for example, have a specific heat of at least about 1.5, more for example, at least about 1.6, when they are in the state at the elevated temperatures.

A perspective posterior view and an anterior view of an embodiment of the handle portion 12 are shown in FIGS. 13 and 14, respectively. At the distal end of the handle may be an end cap 30, including, according to one embodiment, electrical contacts 31, 32, 33 so that the curing light may be seated in a charger base (shown in FIGS. 10 and 11) for recharging the battery 70, if the curing light is battery powered. The end cap 30 and/or the charger base (as exemplified in FIG. 10), may also be so constructed as to provide means for diverting heat away from the curing light after use.

The housing, including its handle portion 12 and front portion 14, may be constructed of a high temperature polymer or composite, such as ULTEM®., which is an amorphous thermoplastic polyetherimide or Xenoy®. resin, which is a composite of polycarbonate and polybutyleneterephthalate or Lexan®. plastic, which is a copolymer of polycarbonate and isophthalate terephthalate resorcinol resin, all available from GE Plastics, or any other suitable resin plastic or composite. At the same time, high impact polystyrene, some polyesters, polyethylene, polyvinyl chloride, and polypropylene may also be suitable.

Polymeric composites, as mentioned above, such as engineering prepregs or composites are also suitable for the composition of the housing. The composites may be filled composites, filled with conductive particles such as metal particles or conductive polymers to aid in the heat dissipation of the device.

An on/off button or switch 18 may be located on the handle portion 12, near the junction between the handle portion 12 and the front portion 14, for manually turning on/off of the curing light. The button may be a molded part, made out of a polymer such as high temperature plastics or polymers used in other parts of the housing, as discussed above. It may also be of the same or different color from the housing. A different color may also help to accentuate its presence and make it easier to find.

LCD screen 500 may also be located on the handle portion 12. LCD screen 500 can be used to display information about the procedure or the curing light device 10 itself. For example, some information that may be displayed can include light wavelength, runtime, battery charge left and a timer. In addition, function buttons 502 can be used to control different aspects of the device. For example, the buttons can control the procedure type, light wavelength, timer settings, and any other function that the curing light device 10 can perform. Additionally, LCD screen 500 can be a touchscreen allowing the user to control the curing light device 10 functions by interfacing directly with the LCD screen 500 and making function buttons 502 unnecessary.

It is also worth to mention that the electrical and control components may be received within the housing portions 12 and 14 towards the distal end 13 of the curing light 10. The curing light 10 may be battery powered or tethered to a power source or a transformer. Battery powered curing lights may offer better portability.

Referring to FIG. 3, a battery 70 may provide electrical power for operating the light source 20 via battery contacts 70a and pin connector 40. In one embodiment, a single rechargeable battery such as a lithium ion battery may be used to power the curing light 10. The on/off button 18 may serve to manually operate the curing light by providing a user input signal through a shaft or post 17, which interfaces with a printed control circuit board 50, may also be located within the handle portion 12, and is mounted close to the battery 70, for example. In one embodiment, a control circuit board 50 includes a device, which may or may not include a microprocessor, which monitors battery life, LED temperature, or system functionality. The battery can be removable or non-removable and mounted inside of the curing light device 10 or be mounted externally.

The end cap 30 is cylindrical in shape and may be attached to the distal end of the handle portion 12. It may be molded as part of the handle portion 12. It may also be attached by other means, such as adhesive bonding, heat bonding, or threaded attachment.

In one embodiment of the invention, as shown in FIG. 10, the charger base may include an electric motor mechanically coupled to a fan or turbine 201. The fan or turbine 201 may be adapted to draw or urge ambient air across a surface of the heat sink 60 to provide cooling of the heat sink 60. In one embodiment, this cooling may occur when the curing light is at rest or being recharged. In another embodiment, the cooling means is present inside a charger base or cradle 200, for recharging the curing light. In other embodiments, the charger base or cradle 200 may not have a fan 201 or cooling means, but instead or additionally, many include a display panel (not shown) for displaying a condition of the battery. In another embodiment, the heat dissipation device may include a compressed air cooling system.

Still referring to FIG. 10, a separate battery charger module 2200 is included in one embodiment. The charger module 2200 is adapted to receive AC power into a plug 400 from a traditional wall socket and provide DC power to the curing light for battery charging.

The battery charger module 2200 illustrated in FIG. 10 has a cable 428 for conducting electricity from the plug 400 to the charger module 2200. The battery charger module 2200 includes circuitry 430 for controlling battery charging of batteries.

FIG. 15 is an alternative embodiment to the invention, which is a dental curing light in a “gun” configuration. The gun style curing light device 600 will have a housing 602. On the housing 602 is LCD screen 500. The housing will also have intake vents 501. Extending from the housing is tip 603. Handle portion 601 is angled away from housing 602. The angle at which handle portion 601 protrudes from housing 602 is such that the user can hold handle portion 601 and manipulate the on/off button or switch 18 and function buttons 502 comfortably without needing to change hand position.

Tip 603 can be removable. Exit vent 506 will be located near the end of the tip, so that air exiting the gun style curing light device 600 will be directed towards the teeth being exposed by the curing light.

The light source can be any number of light sources known to those skilled in the art, such as, but not limited to lamps, LEDs and fiber optic bundles. The light source can be located within housing 602, with tip 603 acting as a light guide to direct the light towards the teeth to be treated. On the other hand, the light source can be mounted at the end of tip 603.

LCD screen 500 can display information about the procedure or the gun style curing light device 600 itself. The function buttons 502 can be used to control different aspects of the device. The LCD screen 500 can be a touchscreen as an alternative to using function buttons 502.

The fan module 503 that is located within housing 602 will work as shown in FIG. 5. Air is drawn into housing 602 through the intake vent 501. At least one intake vent 501 will located on the housing 602, although more intake vents 501 can be added. Air drawn from the intake vents 501 pass through the intake channel 504 to the fan 507. Fan 507 will then blow the air through the output channel 505 where the air exits the gun style curing light device 600 through exit vent 506 located at the end of tip 603.

Handle portion 601 will contain rechargeable battery 70. In addition, heat sink 60 will be located either in the handle portion 601 or the housing 602. Heat sink 60 can be any number of types of heat sinks, such as a phase change heat sink, a fan or a solid metallic block.

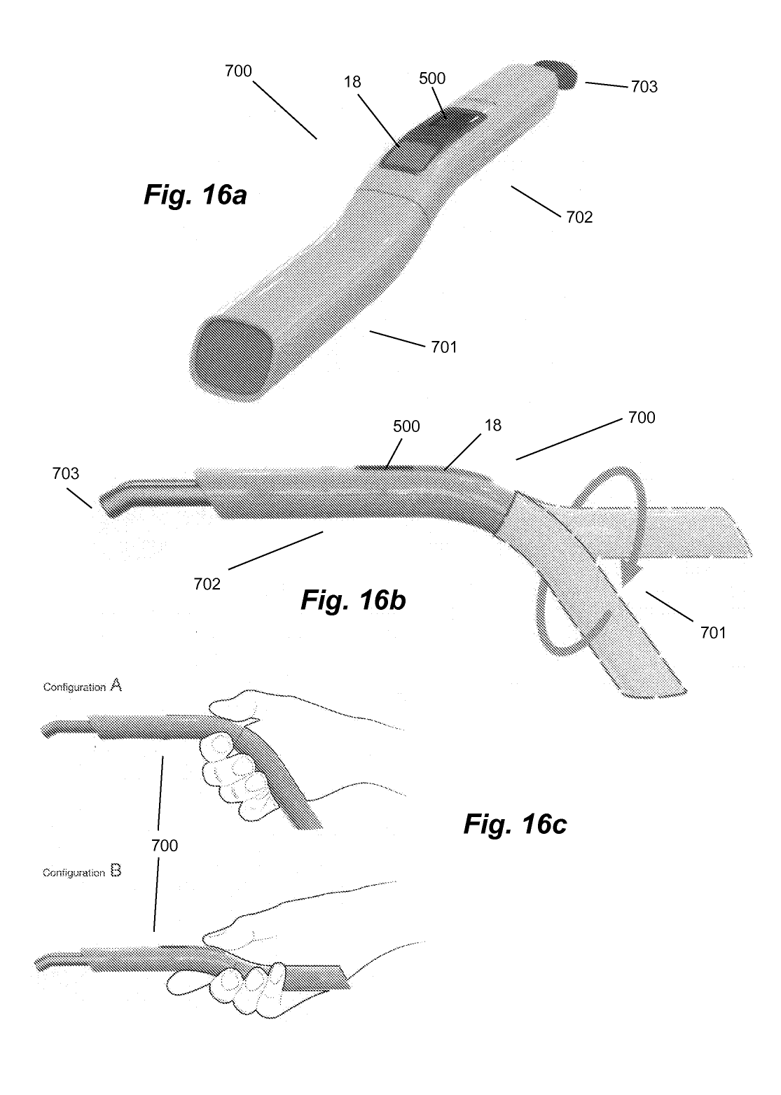

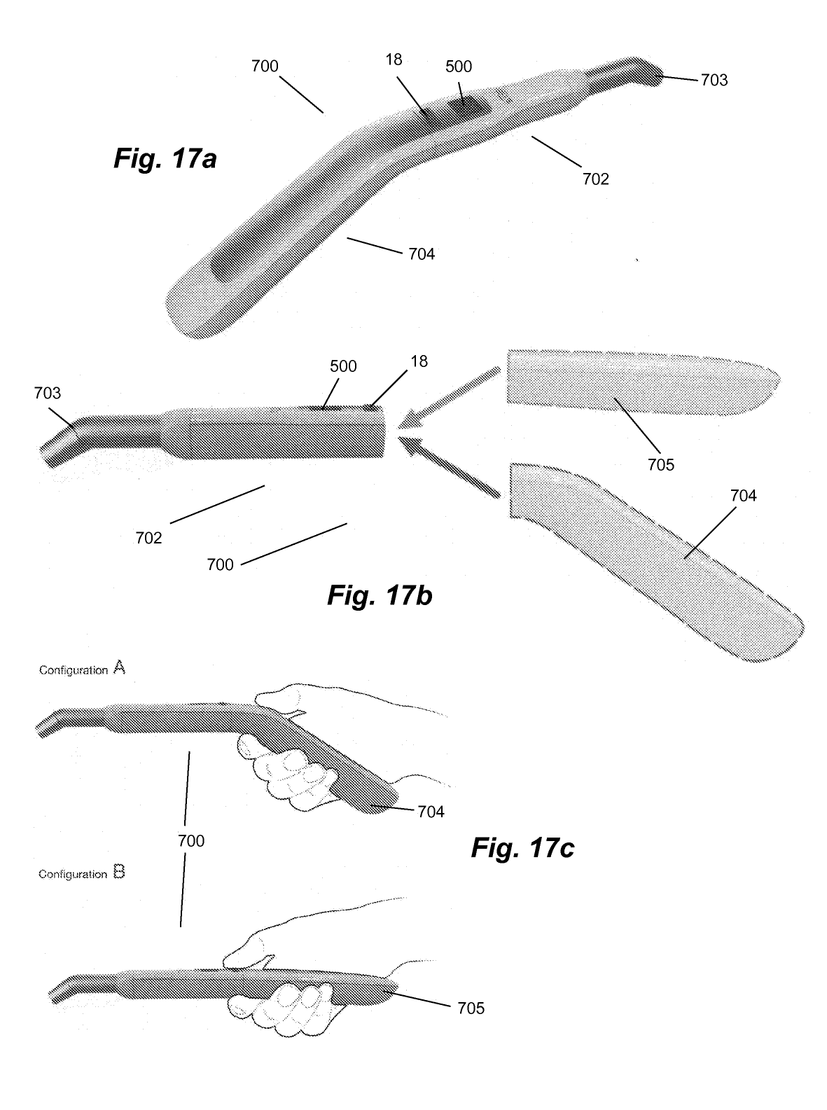

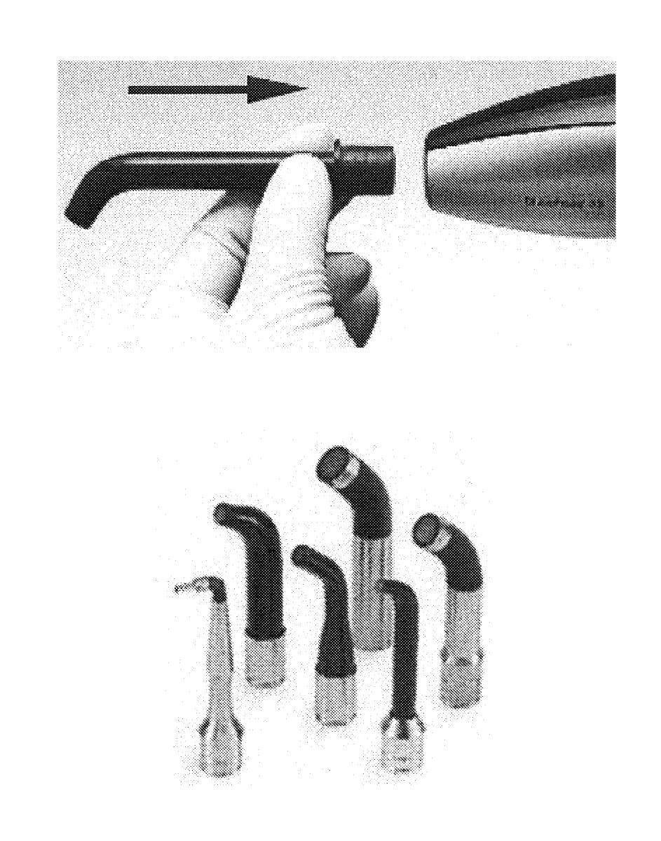

FIGS. 16a-17c is yet another alternative embodiment to the invention where the curing can be converted between a “pen” configuration and a “gun” configuration. FIGS. 16a and 17a shows convertible curing light device 700. LCD screen 500 displays information relevant to the procedure or the status of the convertible curing light device 700. LCD screen 500 is on the housing 702. Also located on housing 702 is on/off button or switch 18. On/off button or switch 18 is located on housing 702 such that the user can operate it easily when the convertible curing light device 700 is either in the pen or gun configuration.

Tip 703 extends from housing 702. Tip 703 can be removable. Exit vent 506 will be located near the end of the tip, so that air exiting the convertible curing light device 700 will be directed towards the teeth being exposed by the curing light.

The light source can be any number of light sources known to those skilled in the art, such as, but not limited to lamps, LEDs and fiber optic bundles. The light source can be located within housing 702, with tip 703 acting as a light guide to direct the light towards the teeth to be treated. On the other hand, the light source can be mounted at the end of tip 703.

The fan module 503 that is located within housing 702 will work as shown in FIG. 5. Air is drawn into housing 702 through the intake vent 501. At least one intake vent 501 will located on the housing 702, although more intake vents 501 can be added. Air drawn from the intake vents 501 pass through the intake channel 504 to the fan 507. Fan 507 will then blow the air through the output channel 505 where the air exits the convertible curing light device 700 through exit vent 506 located at the end of tip 703.

In the convertible curing light device 700 embodiment that is displayed in FIG. 16a, handle 701 is rotatably coupled to housing 702. In FIG. 16b, handle 701 rotates with respect to housing 702. By rotating handle 701, the angle of handle 701 with respect to housing 702 changes such that the convertible curing light device 700 changes from being held as a “gun” style curing light device to a “pen” style curing light device. FIG. 16c shows Configuration A of convertible curing light device 700 as being a “gun” style curing light device and Configuration B of convertible curing light device 700 as being “pen” style curing light device following the handle 701 rotation shown in FIG. 16b.

In the convertible curing light device 700 embodiment that is displayed in FIG. 17a, gun style handle 704 is removably coupled to housing 702 and interchangeable with pen style handle 705. FIG. 17b, shows that gun style handle 704 and pen style handle 705 are attached to housing 702, depending on the user's desired configuration. FIG. 17c shows Configuration A of convertible curing light device 700 with gun style handle 704 attached to housing 702. FIG. 17c show Configuration B of convertible curing light device 700 as being a “pen” style curing light device with pen style handle 705 attached to housing 702.

FIG. 18 discloses an embodiment where the removable tip is a smart tip 550. Smart tip will have a distal end 551 and a proximal end 552. Proximal end 552 is removably coupled to the housing 602. In the alternative, proximal end 552 can be coupled to housing 702. Proximal end 552 of the smart tip 550 will have a connector 553. Connector 553 corresponds to opening 554 on the housing 602. The connector 553 and corresponding opening 554 can utilize any number of removable attachment means known to those skilled in the art such as, but not limited to, friction fit, a screw and nut arrangement, or a pin and jack arrangement.

Housing 602 will contain a sensor which can determine certain parameters of the smart tip 550 when it is inserted into the housing 602. Some of the detectable smart tip 550 parameters includes, but is not limited to, diameter, length, curvature and shape. The sensor can be, but is not limited to, mechanical, optical or magnetic. For example, one possible embodiment is that the connector 553 is comprised of an arrangement of pins. The sensor in housing 602 will identify the parameters of the smart tip 550 based on the pin arrangement that it detects when the smart tip 550 is attached to housing 602.

The advantage of the curing light device being able to detect the parameters of the smart tip 550 that is attached to it is that the curing light device can adjust the power output based on the parameters of the smart tip 550. If the curing light device does not adjust the power output based on the parameters of the tip that is attached to it, then light output will vary according to the tip that is attached to the curing light device. Generally, if the radiometric power on the input side of the tip remains constant, light output per unit area will increase as the tip size decreases. The problem that results is that dental professionals may end up using a tip which emits the proper area light spot, but the radiometric power is not ideal.

However, since the smart tip 550 will communicate the parameters of the tip to the device, the device can automatically adjust the power output depending on the smart tip 550 attached to the housing 602. The advantage to that is that the light output per area will remain consistent no matter which smart tip 550 is attached to the housing 602. Another benefit is that the battery life of the device will be extended. The battery life is extended because certain smart tip 550 parameters will utilize a lower power draw to project the same constant output. For example, a tip with a smaller diameter will draw less power to have the same constant output as a tip with a larger diameter.

In another embodiment, a variety of smart tips 550, covering a wide range of parameters could be packaged together as a kit. FIG. 18a is a sample arrangement of smart tips 550 which may be included in a smart tip 550 kit. In this sample arrangement of smart tips, each tip has a different diameter:

| | Diameter |
|---|---|
| 550a | 2 mm |
| 550b | 8 mm |
| 550c | 9 mm |
| 550d | 13 mm |
| 550e | 4 mm |
| 550f | 11 mm |

It is important to note that smart tips 550 are not limited to the aforementioned diameters, this listing is for illustrative purposes only. A kit can contain any number of smart tips 550 covering any combination of smart tip 550 parameters.

Having described the invention by the description and illustrations above, it should be understood that these are exemplary of the invention and are not to be considered as limiting. Accordingly, the invention is not to be considered as limited by the foregoing description, but includes any equivalents.

1. A curing light device comprising:
a housing comprising a distal end and a proximal end;
a light source mounted within said housing;
an attachment means at said proximal end of said housing;
a tip that directs light away from said light source towards a patient's mouth is removably coupled to said attachment means;
wherein said tip has an identification means for identifying at least one parameter of said tip; and a sensor located within said housing in electronic communication with said light source;
wherein said sensor identifies said at least one parameter of said tip from said identification means and adjusts the light output per unit area of said light source based on said parameters of said tip.

2. The curing light device of claim 1 wherein said at least one parameter that said sensor identifies is selected from the tip diameter, the tip length, the tip curvature, the tip shape and combinations thereof.

3. The curing light device of claim 1 wherein said housing is in a pen configuration.

4. The curing light device of claim 1 wherein said housing is in a gun configuration.

5. The curing light device of claim 1 wherein said housing can convert between a pen configuration and a gun configuration.

6. The curing light device of claim 1 wherein said tip further comprises a male connector.

7. The curing light device of claim 6 where said attachment means further comprises a female connector.

8. The curing light device of claim 7 wherein said sensor determines said at least one parameter of said tip based on said male and female connecting.

9. The curing light device of claim 1 further comprising a heat sink.

10. The curing light device of claim 9 wherein said heat sink is a phase change heat sink.

11. The curing light device of claim 1 wherein said identification means comprises of electrical, magnetic, optical, mechanical and combinations thereof.

12. The curing light device of claim 1 where said sensor comprises of electrical, magnetic, optical, mechanical and combinations thereof.

13. A kit for curing light curable materials comprising:
a curing light comprising of;
a housing comprising a distal end and a proximal end;
a light source mounted within said housing;
an attachment means at said proximal end of said housing;
a sensor located within said housing in electronic communication with said light source; and
a plurality of removable tips with an identification means that removably couple at said attachment means;
wherein each individual removable tip has different parameters; and said sensor identifies said parameters from said identification means on said tip and adjusts light output per unit area from said light source based on said parameters of said removable tip inserted into said curing light such that output remains constant despite the different parameters of said plurality of removable tips.

14. The kit of claim 13 wherein said parameters are selected from the tip diameter, the tip length, the tip curvature, the tip shape and combinations thereof.
